(19) United States
(12) Patent Application Publication
(10) Pub. No.: US 2007/0012324 A1
(43) Pub. Date: Jan. 18, 2007

(54) Handheld Dedicated Device for Diabetes Management

(76) Inventors: Ratna Nirkondar, Pleasanton, CA (US); Giri Nirkondar, Pleasanton, CA (US)

Correspondence Address: WHITE-WELKER & WELKER, LLC, P.O. BOX 199, CLEAR SPRING, MD, 21722-0199, US

(21) Appl. No.: 11/458,041

(22) Filed: Jul. 17, 2006

Related U.S. Application Data
(60) Provisional application No. 60/700,651, filed on Jul. 18, 2005.

(51) Int. Cl. A61B 19/00

(52) U.S. Cl. 128/898; 600/300

(57) ABSTRACT

Handheld dedicated device for diabetes management. The device has nutritional information for 35,000 food items. Food items from fast food restaurants and various brands of food. The device provides a way of looking up all this information. The device can store information entered by the user of the device on food eaten, glucose levels, medication, insulin and exercise data. It also provides a way to save information from blood tests like Hemoglobin A1c which is an average of blood glucose levels over three months, blood pressure, and cholesterol. The input source for the device is keypad or voice command means.

### BACKGROUND OF THE INVENTION

Those diagnosed with diabetes, know that much of their long-term health depends on good diabetes diet. Tracking one's diabetes diet, nutritional information, and exercise to manage glucose levels may seem like a lot of work, but if left untreated, diabetes can lead to serious complications. The most common diabetes symptoms are increased urination, thirst, hunger and fatigue, as well as blurry vision, irritability, and weight loss.

Education about what factors can influence one's blood sugar because a well-balanced diabetes diet can have a huge positive impact on one's health. Planning well-balanced meals and exercising regularly can help keep blood sugar level as close to normal as possible—which will have a positive short term impact and help avoid complications in the future.

When food is eaten it is converted to glucose. Cells then use insulin to convert that glucose into energy. When the body doesn't produce enough insulin or doesn't properly use the insulin it does produce, blood glucose levels will be high because the glucose is not getting into the cells where it is needed.

While the cause of diabetes is unknown, genetics plays a role and obesity and exercise are also key factors. People with pre-diabetes symptoms can reduce their risk of developing diabetes by following good diabetes nutrition, losing weight if necessary, and getting moderate amounts of exercise.

Type 1 diabetes is when the body does not produce enough insulin. Type 2 diabetes, the most common form, is when the body does not properly use the insulin it produces. Gestational diabetes develops in about 4% of pregnant women and can last through the pregnancy. A woman who has had gestational diabetes is more likely to develop Type 2 diabetes later in life.

In pre-diabetes, blood glucose levels are higher than normal, but not high enough to be characterized as diabetes. The risk of developing diabetes, however, is quite high. Again, good diabetes nutrition and diet, with modest weight loss and moderate physical activity is the key. Often people with pre-diabetes can delay or prevent Type 2 diabetes by adopting healthier lifestyles.

The views on what a good diabetes diet means have undergone numerous revisions. One should, of course, consult their health care advisor for details about a recommended diabetes diet. In general, good diabetes nutrition involves being aware of the nutritional information of what you eat and its impact on your blood glucose. Typically one needs to balance and track food, medication, and exercise. Eating regularly, and on time, helps to avoid low-blood glucose. Learning about body mass index and the glycemic index of foods can also be helpful.

The American Diabetes Association recommends keeping records of everything that can affect blood glucose levels, and record the results. This includes recording when and what one eats, when and how much they exercise, insulin or other medication doses, as well as blood checks. Many people begin keeping records in small notebooks. That can be cumbersome, however, and one doesn't always know the nutritional content of what you're eating.

What is needed is a handheld device that helps keep track of diabetes nutritional information. Looking up nutritional information about food, keeping track of what one eats, and recording ones exercise and medication are important parts of diabetes management.

What is needed is a device small enough so one can easily carry it with them wherever they go. The better records one keeps, the better lifestyle they can enjoy.

What is needed is a device that assists with diabetes management that's small enough to slip into a pocket or purse. In addition to providing a quick and easy method of recording one's food, exercise, and medications, specific nutritional information for over 35,000 food items is desired with the ability to create lists to make entering favorite foods easier.

What is needed is a device that records and generates reports to spot trends and patterns in one's blood readings so adjustments can be made in medication, exercise, or eating. This kind of record keeping provides someone with diabetes with a long-term view of their disease that will in turn help them control the disease and lead a healthier life without complications.

### SUMMARY OF THE INVENTION

The present invention is a handheld dedicated device for diabetes management that provides means for the recording of all factors associated with managing diabetes like food intake, exercise, blood glucose readings, blood pressure, hemoglobin A1c levels, medications, insulin intake and cholesterol. Additionally, the device contains nutrition information for over 35,000 food items including packaged foods and prepared foods from major restaurants. Information including calories, fat carbohydrates, protein, fiber, and sodium and exercise data is provided. In addition to tracking food, exercise, sugar and/or insulin details for diabetics, the device can also track blood pressure details for those with a chronic blood pressure problem. The input source for the device is keypad or voice command means.

It is therefore an objective of the present invention to create a handheld device to reduce the risk of complications from diabetes, by tracking all the things medical professionals stated need tracking—everything one eats, when and how one exercises, doses and timings of medications/insulin, blood sugar levels—which can quickly become an overwhelming task without an assisting device. Whether one has Type 1, Type 2, or gestational diabetes, a diabetes management planner can be a crucial ingredient for one's health and diabetes management.

It is another objective of the present invention to create a device that allows one to look up nutrition information of meals, adjust the number of servings, log when and what was eaten, note glucose levels, monitor exercise, and track oral medications and insulin. With the device of the present invention one can easily and accurately track what is need for their specific diabetes management. Additionally one can share records and reports with their health care providers. For example, with the present invention one can print or email reports to their healthcare team—Connect their diabetes planner to their PC via an optional USB connector so data can be downloaded to a PC. Reports can be shared with doctors, dietitians and educators so they can adjust diabetes management goals for their patients with Type 1, Type 2, or gestational diabetes.

It is yet another objective of the present invention to create a handheld device that fits easily into a pocket or purse so one can record dietary information and log exercise routines wherever and whenever they want. Recording information immediately rather than relying on memory or scribbling things down on scraps of paper is much more efficient and accurate. Entering blood glucose readings, insulin, medication, exercise, and meal information can be performed in real-time.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is an exploded view of the device of taught by the present invention;

FIG. 2 is an orthogonal top view of the device of taught by the present invention illustrating the QWERTY keypad, number pad, and display screen;

FIG. 3 is an orthogonal left and right side view of the device of taught by the present invention; and

FIG. 4 is an orthogonal top and bottom side view of the device of taught by the present invention.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

Referring to the figures, it is possible to see the various major elements constituting the device of the present invention. The device consists of an electronic PDA device type dedicated to diabetes management. Both hardware and software work together to help a person manage their diabetes. It is the idea of having a food database and having a lookup so it is easy for a person to get the nutritional content of food before they eat, to keep all the tracking data as related to diabetes in one place, to be able to keep in touch with the healthcare team to communicate all this information to them so they can get better guidance.

Physical Device

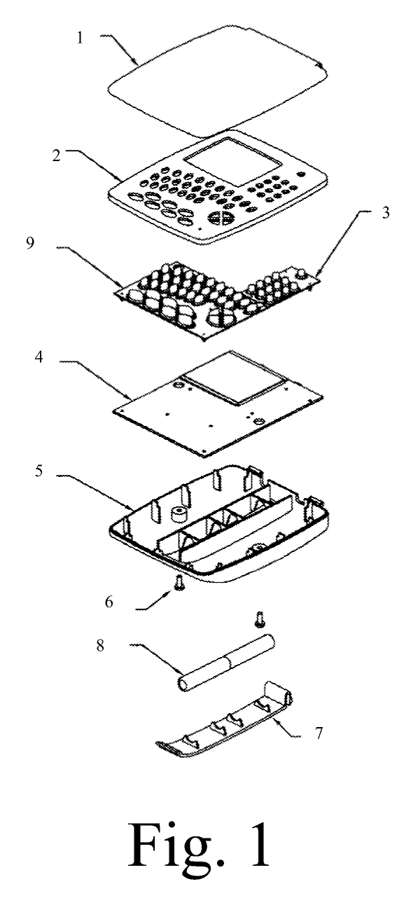

Now referring to FIG. 1, an exploded view of the physical device is illustrated. The device is physically comprised of the following major components assembly in combination: a cover 1, attached to a top housing 2 which covers a QWERTY keypad 9 and number keypad 3 which rests on a PCB assembly 4 that is secured to the a bottom housing 5. The QWERTY keypad 9 and number keypad 3 and PCB assembly 4 are secured between the top housing 2 and the bottom housing 5 by screws 6. Also provided is a battery door 7 that provides easy access to a battery compartment in the bottom housing 5 where the two AA batteries 8 are located which provide power to the device.

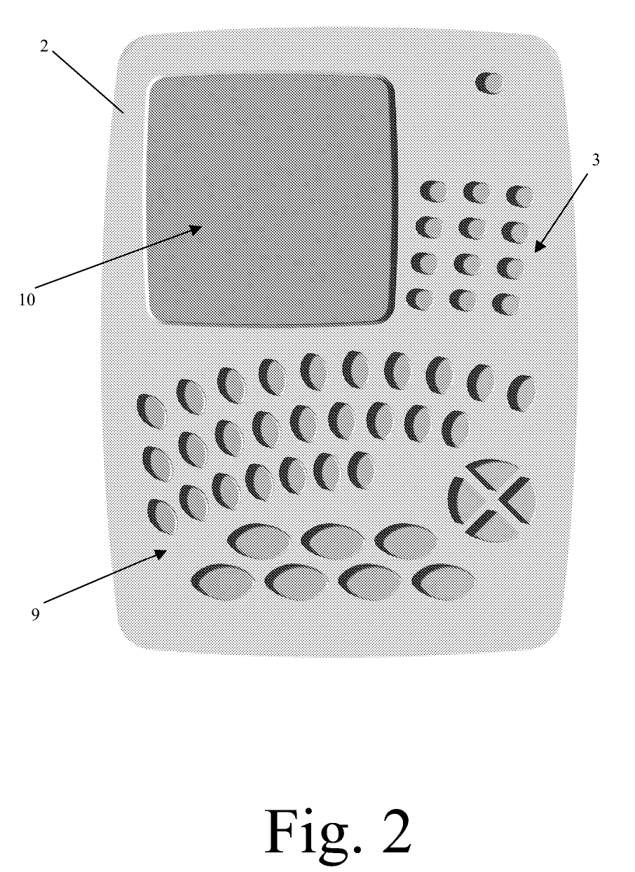

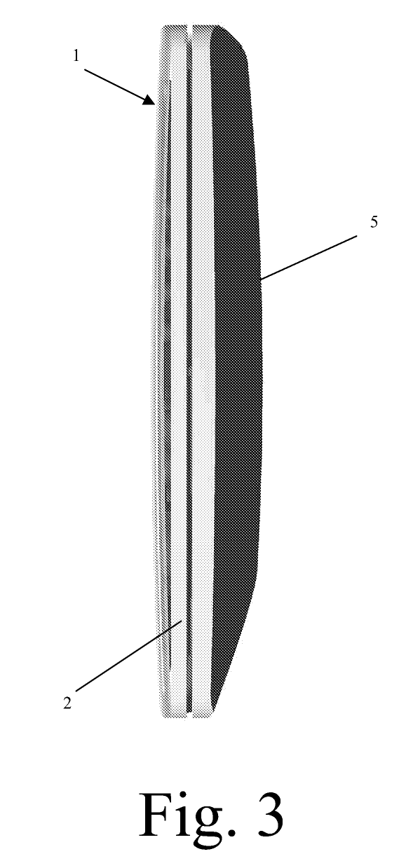

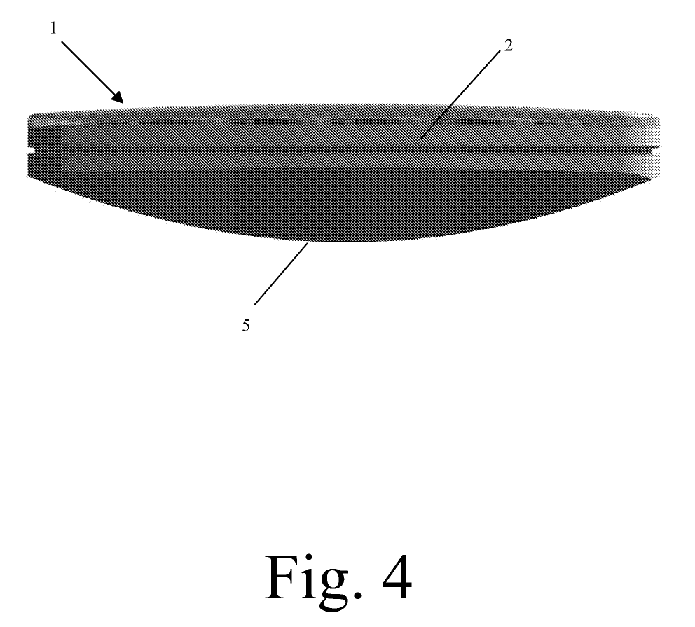

FIGS. 2-4 illustrate orthogonal views of the device of the present invention and illustrate its similarity to other PDA devices known in the prior art. This device of the present invention is distinguishable over prior art devices in that is features a QWERTY keypad 9 below a display screen 10 and a number keypad 3 to the right of the display screen 10.

Electronic Circuit

An electronic circuit that consists of a Main Control Unit (MCU) also known as Central Processing Unit (CPU), memory chips and input and output units is utilized and readily available to one of ordinary skill in the art. The MCU is a general-purpose microprocessor with built-in RAM memory. The memory chips consists of memory to store the user-entered information, to store the device software program and to store the 35,000 food item database containing nutritional information. The input and output units consist of a QWERTY keypad, an LCD display panel and a USB controller used to communicate to a PC via standard USB port. Power to the circuit is supplied from a CR2032 or AAA type batteries.

Transfer Information

Information that is entered by the user in the device is transferred to the computer (windows based PC) by connecting the device to the PC using a USB cable. The transfer protocol consists of the PC sending a command and the device responding to the command.

The command list consists of commands sent by the PC to the device. The command list includes commands sent to test the connectivity and also to request data from the device. The device will respond to each command back with an acknowledgement for connectivity test or with data associated with the command request.

Command List

CS+ To test the connectivity. Device will respond with +:OK:\n
CSH Dump header data from device. Device will respond with a header and data. The header format is: H:NNN:\n where NNN is the number of records. The data format is: H:<<<See User Header Data Format below>>>
CSD Dump user data from device. Device will respond with a header and data. The header format is: D:NNN:\n where NNN is the number of records. The data format is: D:<<<See User Data Format below>>>
CST Dump System Date and Time (Device date and time). Device will respond with the current date and time. The data format is: T:MMDDYYYYHHMM:\n

It is important to note that each command will be terminated with a linefeed (“\n”) character (Decimal 10, Octal: 012)

With respect to the Header Format, each field will be delimited by the character “:”. Each line will be terminated with a linefeed “\n” character (Decimal 10, Octal: 012).

TABLE 1
Format of data types

| Name | Description | Size | Range/Format |
|---|---|---|---|
| Type | Type of | 2 Char | TT |
| | Data | | |
| Value | Value | X | VVVV....VVV (variable length) |
| | | Chars | |
| EOF | End of Data | 1 Char | E (“\n” one line-feed character |
| | | | means end of data) |

The data types will include, but are not limited to: Name, Age, Weight, Height, Daily. Calorie Goal, Glucose Ranges, and Meal Time Slots. For each of the data types the following value fields will respectively be utilized for each. Name, the value field will have the name of the user. For example, the entry will look like this for user John Smith: 01:John Smith:\n. Age, The value field will have the age of the user in years. For example, the entry will look like this for 53 years: 02:53:\n. Weight, The value field will have the weight of the user in pounds (lbs). For example, the entry will look like this for 200 pounds: 03:200:\n. Height, the value field will have the height of the user in inches. For example, the entry will look like this for 70 inches: 04:70:\n. Daily Goals, the value field will have the daily goal for food and exercise 05:NexxxxNCxxxxNFxxxxNIxxxxNSxxxxNPxxxxECxxxxETxxxx:\n.

With respect to Glucose Ranges, the value field will have the glucose ranges defined by the user in the device. For example, the entry will look like this: 06:VxxxxHxxxxLxxxx:\n. Where: V equals very high, glucose values above this value are Very High (value>V); H equals High, Glucose values above H to V are high (value>H AND value<=V); Glucose values below H and above L is in target (value>=L AND value<=H); and L equals Low, glucose values below this value are Low (value<L) xxxx=four digit number padded with zeros.

Finally, for Meal Time Ranges, the value field will have the meal time ranges defined by the user in the device. For example, the entry will look like this: 07:BFhhmmMShhmmLUhhmmAShhmmDIhhmmDShhmm:\n where BF is a code for Breakfast Start time, MS is a code for Morning Snack start time, LU is a code for Lunch Start time, AS is a code for Afternoon Snack Start time, DI is a code for Dinner Start time, DS is a code for Post Dinner Snack Start time, and hhmm equals hour and minute (fixed length).

Codes will be displayed and used in the information transfer step and include those as listed in Table 2.

TABLE 2
Codes

| | |
|---|---|
| NE | Calories (Energy) |
| NC | Carbohydrates (grams) |
| NF | Fiber (grams) |
| NI | Total Fat (grams) |
| NS | Sodium (milligrams) |
| NP | Protein (grams) |
| EC | Exercise Calories |
| ET | Exercise Time spent (minutes) |

User Data Format

With respect to user data format and data types as shown in Table 3, each field will be delimited by the character “:”. Each line will be terminated with a linefeed “\n” character (Decimal 10, Octal: 012). For each of the data types the following value fields will respectively be utilized for each. Food, the value field will have the name of the food AAAAA followed pairs of the format NN FFFFFFFF. The format is “AAAAA”,“SSSS”,NNFFFFFFFF. Where AAAA is a variable length text string of food name; SSSS is a variable length text string that contains number of servings. NN is the two character nutrient code (calories, carbohydrates, protein, etc) and FFFFFFFF is the eight-character value for the nutrient code (padded with zeros). AAAA is enclosed in quotes and separated by a comma from the serving size SSSS. SSSS is enclosed in quotes and separated by a comma from food name and value pairs.

Nutrient Codes include: NE, Calories (Energy); NI, Total Fat; NX, Saturated Fat; NM, Monounsaturated fat; NP, Protein; NC, Carbohydrates; NG, Sugars; NF, Fiber; NH, Cholesterol; NS, Sodium; and NT, Trans fatty acids. For example, the entry will look like 02:04292004:1050:“Snack”,“1”,NE00000080NF00000009: This is a comment:\n. The above entry is for food made on 10:50 am Apr. 29, 2004. The name is “Snack” and the number of servings is 1. The calories (NE) is “80”, fat (NF) is “9” and comment is “This is a comment”.

Food By Calories, the value field will have pairs of the format NNFFFFFF. Where NN is a two character nutrient code (calories, carbohydrates, protein, etc); FFFFFFFF is an eight-character value for the nutrient code (padded with zeros). For example, the entry will look like 02:04292004:1050:NE00000080NF00000009:This is a comment:\n. The above entry is for food (02) made on 10:50 am Apr. 29, 2004. The calories (NE) is “80”, fat (NF) is “9” and comment is “This is a comment”.

Water Consumed, the value field will have a string of the format WWW where WWW is the amount of water consumed in milliliters (ml). For example, the entry will look like 03:04292004:1050:250:One cup of water\n. The entry is for one cup of water (250 ml) made at 10:50 am Apr. 29, 2004. The value is “150” and comment is “One cup of water”.

Exercise, the value field will have a string of the format “EEEE”,NNMMNNCCCC where: EEE is the type of exercise as a variable text string; NN is the code for the value; MM is the number of minutes exercised (fixed length of 2); CCCC is the amount of calories consumed (fixed length of 4—padded with zeros); and for NN: EM—minutes exercised, and EC—calories burnt. For example, the entry will look like 04:04292004:1050:“Treadmill”,EM30EC0200:After breakfast\n. The entry is for 30 minutes of walking at 10:50 am Apr. 29, 2004. Calories burnt are 200 and comment is “After breakfast”.

Sugar, the value field will have a string of the format SSSS where SSSS is the blood sugar in mg/dl. For example, the entry will look like 05:04292004:1050:120:Post Dinner\n. The entry is for sugar reading of 120 mg/dl at 10:50 am Apr. 29, 2004. The comment is “Post Dinner”.

Ketones, the value field will have a string of the format KK where KK is the code for the level of Ketones: KN means Negative, KT means Trace, KS means Small, KM means Moderate, and KL means Large. For example, the entry will look like 06:04292004:1050:KL:Wake up\n. The entry is for large ketones at 10:50 am Apr. 29, 2004. The comment is “Wake up”.

Medical Record, the value field will have a string of the format WTxxxx.xxACxxxx.xxBSxxxxBPxxxxCTxxxxCLxxxxCHxxxxTRxxxxTHxxxx.xxUCxxxx.xx ALxxxxBMxxxx.xx where WT means Weight (float xxxx.xx), AC means Hemoglobin A1C (float xxxx.xx), BS means systolic BP (integer xxxx), BD means diastolic BP (integer xxxx), CT means Total Cholesterol (integer xxxx), CL means LDL cholesterol (integer xxxx), CH means HDL cholesterol (integer xxxx), TR means Triglycerides (integer xxxx), TH means Thyroid TSH (float xxxx.xx), UC means Urine Creatanine (float xxxx.xx), AL means Albumin (integer xxxx), and BM means Body Mass index (float xxxx.xx).

Medication, the value field will have a string of the format “MMMM . . . ”,“DDDDD” where MMMM is a variable length text string of medication name and DDDDD is a variable length text string of dosage value. The two string will be enclose in quotes(“ ”) and separated by a comma (,). For example, the entry will look like 08:04292004:1050:“Tylenol”,“500mg”: Headache and Allergies:\n. The entry is for taking “500 mg” of “Tylenol” at 10:50 am Apr. 29, 2004. The comment is “Headache and Allergies”.

Insulin, the value field will have a string of the format NNNNDDDD where NNNN is a variable length text string of insulin name and DDDD is a variable length text string of dosage value The two string will be enclose in quotes(“ ”) and separated by a comma (,). For example, the entry will look like 09:04292004:1050:“NPH”,“2 units”:Morning\n. The entry is for taking 2 units of NPH at 10:50 am Apr. 29, 2004. The comment is “Morning”.

TABLE 3
User Data Types

| Field | Description | Size | Range/Format |
|---|---|---|---|
| Type | Type of | 2 Char | TT (Fixed length of 2) |
| | Data | | |
| Date | Date of | 8 Char | MMDDYYYY (Fixed length of 8) |
| | Entry | | |
| Time | Time of | 4 Char | HHMM (Fixed length of 4) |
| | Entry | | |
| Time Slot/ | Time Slot or | 2 Char | SS (Fixed length of 2) |
| Meal Time | Meal Time | | |
| Value | Value | X | VVVV....VVV (variable length) |
| | | Chars | |
| Comments | Comments | X | CCCC...CCC (variable length) |
| | | Chars | |
| EOF | End of Data | 1 Char | E (“\n” one line-feed character |
| | | | means end of data) |

Software for the Personal Computer (PC)

The software for the PC consists of two parts working together as one application. One part is used to communicate to the device and transfer data into a database in the PC. The other part is to analyze the data and produce charts and reports that the user can review or share with their health care provider.

The Device is connected to a PC via USB port. Data received from the device should overwrite the data locally stored. Data cannot be modified from the PC. It can be modified only from the device.

Each time the user connects the device to the PC, his/her personal data should be downloaded. If the user is connected the first time, he/she should be asked to create his/her own repository folder. The software allows the user to save, e-mail, and print any report or chart he/she can see in the program main window. Reports and charts are generated in HTML or Adobe PDF format. Users can save the report in their own repository and can print any report at any time. User cannot modify the report from the PC. Reports that are necessary to send to a doctor are converted to PDF first if not already in PDF format. All reports sent are stored in Repository in a “Sent” folder. The software makes a log of reports sent so users can review the report sent using the software interface, selecting the report from the log. In a first step the software is installed from a software distribution CD on a users computer by itself without the device being attached. The Device can be used for transfer of data anytime independent of the CD after the program is installed. When the USB cable is plugged in and CD put in the computer a program is automatically launched. The following buttons always appear on bottom of the screen: Download data, Add to reports file, email report, print report, fax report.

After every report is generated the user is given an option to add it to a pool of reports. When the user wants to send it to the doctor the user accesses the pool and sends it. Optionally the user should be able to send the report from the screen the user is on. Reports include, but are not limited to: Log List-Report of all details. By date range; Summary Information of food and glucose for date range; Food Information for date range; Total Calories; Food information/Glucose based on meal type; Food information for time range when there was a high; Food information for time range when there was a low; Medication information for date range; Insulin information for date range; Exercise information; Blood Pressure Report; Log List—Report of all details By date range.

Summary Information of glucose for date range includes but is not limited to: Average Blood Glucose; Highest Blood Glucose; Lowest Blood Glucose; Standard Deviation; Number of glucose readings; Days Covered; Number of days without tests; and Average Readings per day.

The software can also generate the following charts: Average glucose per day; Glucose by timeslots; Detail glucose trend for a given time slot; Glucose Pie Chart; Glucose trend over a day; Glucose—Total Effect; Carbohydrates and Protein in take over a day; Insulin intake over a day; and Exercise over a day.

It is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. A Handheld Dedicated Device for Diabetes Management comprising:
a cover;
a top housing;
a QWERTY keypad;
a number keypad;
a PCB assembly with a display screen;
software means stored on said PCB assembly for controlling the device;
a bottom housing;
wherein the QWERTY keypad and number keypad and PCB assembly are secured between the top housing and the bottom housing by screws;
said bottom housing is further comprised of a battery door that provides easy access to a battery compartment within said bottom housing where the two AAA batteries are located to provide the device with power to operate.

2. The device of claim 1 the software means to collect diabetes related information consists of menus and screens navigated with the QWERTY keypad and the software storing information in a flash memory.

3. The device of claim 2 wherein the software running on the PC includes:
a software application on a PC computer connected via USB cable to the device with the software to provide means for data reception from the device, data processing, reports generation, and reports printing and sending via e-mail.

4. The device of claim 3 wherein
said Device is connected to a computer via a USB port;
said Device responds to commands sent by the software on the PC.

5. The device of claim 4 wherein said software allows:
saving, e-mail, and printing any report and the generation of a report log providing means for review of the reports, selecting the report from the log; and wherein
users can save the report in their own repository, each time the user connects the device to the PC, and their personal data is downloaded.

6. The device of claim 5 wherein reports specifically include and combination from the report group listed below:
Log List-Report of all details by date range;
Summary Information of food and glucose for date range;
Food Information for date range;
Total Calories;
Food information/Glucose based on meal type;
Food information for time range when there was a high;
Food information for time range when there was a low;
Medication information for date range;
Insulin information for date range;
Exercise information; and
Blood Pressure Report.

7. The device of claim 5 wherein summary information of glucose for date ranges includes:
Average Blood Glucose;
Highest Blood Glucose;
Lowest Blood Glucose;
Standard Deviation;
Number of glucose readings;
Days Covered;
Number of days without tests; and
Average Readings per day.

8. The device of claim 5 wherein the following charts are generated:
Average glucose per day;
Glucose by timeslots;
Detail glucose trend for a given time slot;
Glucose Pie Chart;
Glucose trend over a day;
Glucose—Total Effect;
Carbohydrates and Protein in take over a day;
Insulin intake over a day; and
Exercise over a day.

9. A system for diabetes management that provides means for the recording of:
Hemoglobin A1c;
blood pressure;
and cholesterol; and
nutrition content of foods eaten including:
calories,
fat carbohydrates,
protein,
fiber, and
sodium; and
exercise data; and
sugar or insulin details for diabetics and blood pressure details for those with a chronic blood pressure problem.

10. The system of claim 9 further comprising a handheld device consisting of:
a cover;
a top housing;
a QWERTY keypad;
a number keypad;
a PCB assembly with a display screen;
a bottom housing 5;
wherein the QWERTY keypad and number keypad and PCB assembly 4 are secured between the top housing 2 and the bottom housing by screws 6;
said bottom housing is further comprised of a battery door that provides easy access to a battery compartment within said bottom housing where the two AA batteries are located to provide the device; and
recoding and tracking data as related to diabetes.

11. The system of claim 10 wherein the handheld device is further comprised of a food database having a lookup feature providing means for obtaining nutritional content of food.

12. The device of claim 11 having means to provide reports, data, and charts to healthcare personnel.

13. The device of claim 11 wherein the input source for the device is a keypad or voice command means.
